(19) United States
(12) Patent Application Publication
(10) Pub. No.: US 2018/0206737 A1
(43) Pub. Date: Jul. 26, 2018

(54) DUAL SENSOR

(71) Applicant: ORIDION MEDICAL 1987 LTD., Jerusalem (IL)

(72) Inventor: Joshua Lewis Colman, Jerusalem (IL)

(21) Appl. No.: 15/874,202

(22) Filed: Jan. 18, 2018

Related U.S. Application Data
(60) Provisional application No. 62/448,468, filed on Jan. 20, 2017.

(51) Int. Cl. A61B 5/0205; A61B 5/00; G01N 33/497

(52) U.S. Cl. CPC A61B 5/0205; A61B 5/0075; A61B 5/02416; G01N 33/497; A61B 5/7242

(57) ABSTRACT

A breath monitoring device including a flow sensor that may measure a respiration rate of a patient and to identify apnea events in the patient's breathing; a CO₂ sensor that may operate at low power and to measure a CO₂ concentration in the patient's breath; and a processor that may integrate the measurements obtained from the flow sensor and the CO₂ sensor and to determine the respiratory status of the patient based on the integration.

This application claims the benefit of U.S. Provisional Application Ser. No. 62/448,634 filed Jan. 20, 2017, the content of which is incorporated herein by reference in its entirety for all purposes.

### TECHNICAL FIELD

The present disclosure generally relates to the field of breath monitoring, specifically to low power, low cost breath monitoring solutions.

### BACKGROUND

Capnographs are used to monitor ventilation, where for this purpose three main outputs of the capnograph shed light on the patient's ventilation status: a) End tidal Carbon dioxide concentrations (EtCO₂)—the highest concentration of CO₂ at the end of the exhalation stage b) CO₂ concentration as function of time, and c) Respiration Rate. Hence, it may be advantageous to provide a method for enabling monitoring and measuring of changes in flow dynamics and CO₂ concentration measurements, while leveling out time delays related to the CO₂ sampling system.

The response time of capnographs may be delayed due, in part, by transit time, i.e. the time taken by the CO₂ to travel (“transit”) from the sampling inlet to the sampling cell and rise time, i.e. how “quickly” the analyzer responds once CO₂ has entered the sampling cell. Much effort has been made to reduce the rise time of capnographs. The rise time of capnographs has been considerably reduced in newer units by using powerful amplifiers, sophisticated sensor technologies, by minimizing the volume of the sampling chamber and by utilizing relatively high sampling flow rates.

### SUMMARY

In order to achieve sensitive and informative CO₂ waveforms, efforts are continuously being made to develop capnographs which achieve the goal of reduced response times (e.g., a response time less than approximately 250 milliseconds (msec)). However, this has resulted in expensive capnography units having high power consumption and reduced mobility, limiting their widespread usage.

Advantageously, the dual sensor breath monitoring device disclosed herein, enables accurate breath monitoring while utilizing low power, cost effective CO₂ monitoring solutions. The dual sensor breath monitoring device, disclosed herein, includes a first sensor (e.g., flow sensor with inherent, fast rise time characteristics) configured to monitor breath flow for the purpose of monitoring and catching the fast-changing ventilation and breathing characteristics of a patient, in conjunction with a second sensor (e.g., a CO₂ sensor, with an inherent slower rise time relative to the flow sensor) configured to measure and capture the characteristic, slower changing CO₂ concentrations of a patient's blood as measured in most cases, but not only via the breath. The CO₂ sensor used hence may be a “low power” CO₂ sensor, either as a result of inherent limitations to the technology, its level of sophistication or by way of how it is activated.

The flow sensor of the dual sensor is responsible for measuring fast changing parameters, such as respiration rate and apnea identification, while the low power CO₂ sensor is responsible for monitoring slow-changing parameters, such as ventilation efficiency and the body's ability to maintain CO₂ concentrations required for maintaining a correct blood pH. The conjunction of the “fast response” flow sensor and the “slow response” CO₂ sensor advantageously provides a reliable, cost effective and easily portable system suitable for implementation in out-of-hospital (e.g. ambulances), as well as in areas of low and/or limited resources.

According to some embodiments, there is provided a breath monitoring device including a flow sensor configured to measure a respiration rate of a patient and to identify apnea events in the patient's breathing, a CO₂ sensor configured to operate at low power and to measure a CO₂ concentration in the patient's breath; and a processor configured to integrate the measurements obtained from the flow sensor and the CO₂ sensor and to determine the respiratory status of the patient based on the integration.

According to some embodiments, the CO₂ sensor may include a blackbody, a molecular correlation spectroscopy (MCS) source, an infrared (IR) LED, or any combination.

According to some embodiments, the CO₂ sensor may be configured to operate intermittently and/or to operate at a modulation frequency below 20 Hertz (Hz) and at a duty cycle below 50 percent. For example, in order for the CO₂ sensor to reach reduced response times (e.g., a rise time less than 250 msec), a 20 Hz signal (new reading frequency) may be necessary, where 20 Hz, will provide a signal/reading once every 50 msec. At the reduced response times, at least 4 to 5 readings may be taken in order to capture the graphic changes of the CO₂ concentration in the breath cycle. Generally, sensors which dictate modulation of operation operate at a 50% duty cycle (e.g., ON 50% of the time and OFF 50% of the time). However, to save power, the ON time of the sensor may be reduced, as long as the readings from the sensor are qualitative and provide sufficient information regarding the CO₂ concentration in the patient's breath during the period that the sensor is ON (relative to how fast the sensor can turn on and reach full sensitivity). For example at 20 Hz, signal capturing and 50% duty cycle, the ON time and OFF time of the sensor may be 25 msec and the average power of the sensor is 50%. Reducing the duty cycle of the sensor to save power results in a reduced ON time of the sensor. If the ON time of the sensor is below 25 msec, the quality of the signal may be unsuitable to obtain an accurate reading of the CO₂ in the patient's breath due, in part, to the sensor not having sufficient time to reach signal equilibrium (e.g., reach full sensitivity). Therefore, the ON time of the sensor may need to be at least 25 msec. However, if the signal frequency is reduced from 20 Hz to 10 Hz, (e.g., 50 msec ON and 50 msec OFF at 50% duty cycle), the duty cycle may be reduced to 25%, thereby saving half the power. The ON time of the sensor may remain at 25 msec, which is sufficient to receive a full signal and reading with less information (e.g., 10 readings a second) and at half the energy.

According to some embodiments, the CO₂ sensor may operate intermittently. According to some embodiments, the intermittent operation of the CO₂ sensor may include turning the device and/or the CO₂ sensor ON for a first period of time and turning the device and/or CO₂ sensor OFF for a second period of time, wherein the second period of time is longer than the first period of time.

According to some embodiments, the second period of time is at least twice the length of the first period of time. According to some embodiments, the first period of time may be in the range of about 5-15 seconds and the second period of time may be in the range of about 40-120 seconds.

According to some embodiments, the CO₂ sensor may operate at a modulation frequency below 20 Hz and at a duty cycle below 50 percent, when in use.

According to some embodiments, the modulation frequency of the CO₂ sensor may be determined by the respiration rate monitored by the flow sensor.

According to some embodiments, the CO₂ sensor may operate at a modulation frequency above 20 Hz and at a duty cycle above 40 percent during a first period of time and at a modulation frequency below 20 Hz and at a duty cycle below 40 percent during a second period of time, when in use. According to some embodiments, the second period of time may be longer than the first period of time. According to some embodiments, the second period of time may be at least twice the length of the first period of time. According to some embodiments, the first period of time may be in the range of about 5-15 seconds and the second period of time may be in the range of about 40-120 seconds. The first and second time periods may be updated periodically based on a respiratory condition of the patient (e.g., asthma, apnea), one or more respiratory parameters have been met (e.g., desired respiration rate and/or CO₂ concentration levels are achieved).

According to some embodiments, the CO₂ sensor may be an acoustic sensor configured to measure changes in the speed of sound in a breath sample obtained from the patient. According to some embodiments, the CO₂ sensor may be or include a colorimetric sensor having a membrane and/or dye configured to change its color in response to a change in the pH of a breath sample obtained from the patient. According to some embodiments, the colorimetric sensor may include a detector configured to detect the color of the membrane and/or dye. According to some embodiments, the detector may be an RGB light detector.

According to some embodiments, the colorimetric sensor may be configured to be positioned in the patient's airway.

According to some embodiments, the CO₂ sensor may be or include a transcutaneous CO₂ sensor. According to some embodiments, the transcutaneous CO₂ sensor may be configured to operate periodically, thereby mitigating effects of heat from the sensor on the patient's skin.

According to some embodiments, the CO₂ sensor may be or include a photoplethysmography (PPG) sensor configured to determine the patient's pulse waveform. According to some embodiments, a processing unit may be configured to indirectly measure the patient's arterial CO₂ concentration (PaCO₂), based on a resistance to blood flow as determined from a shape of the pulse waveform obtained from the PPG sensor.

According to some embodiments, the flow sensor may be a pressure type transducer, a thermal type transducer, or any combination thereof.

According to some embodiments, there is provided a method for monitoring a patient's respiratory status, the method including obtaining a respiration rate and/or indications of an apnea event from a flow sensor; obtaining a CO₂ concentration in the patient's breath from a CO₂ sensor, integrating, using a processor, the measurements obtained from the flow sensor and the CO₂ sensor; and determining the respiratory status of the patient based on the integration.

According to some embodiments, the CO₂ sensor may be configured to operate at a low power as essentially described herein.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with the same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 illustrates a dual sensor breath monitoring device including a flow sensor and a low power CO₂ sensor, according to some embodiments;

FIG. 2, illustrates an all optic, breath monitoring device including a fiber optic flow sensor and a calorimetric CO₂ sensor; and

FIG. 3 is an illustrative flowchart of a method for determining a patient's ventilatory status using a dual sensor breath monitoring device, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. Additionally, it is to be explicitly understood that any combination of any one or more of the disclosed embodiments may be applicable and is within the scope of the disclosure.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present disclosure may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of non-transitory memory media suitable for storing electronic instructions, and capable of being coupled to a computer system bus.

The processes and displays presented herein are not inherently related to any particular computer or other apparatus. Various general purpose systems may be used with programs in accordance with the teachings herein, or they may prove convenient to construct a more specialized apparatus to perform the desired method. The desired structure for a variety of these systems will appear from the description below. In addition, embodiments of the present disclosure are not described with reference to any particular programming language. It will be appreciated that a variety of programming languages may be used to implement the teachings of the embodiments as described herein.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

The present disclosure generally relates to the field of breath monitoring, specifically to low power, low cost breath monitoring solutions, which may serve as supplemental solutions to capnography for the purpose of monitoring patient ventilation. The breath monitoring solutions disclosed herein include advantages over conventional capnography in relation to power, simplicity, cost (including cost of ownership), size and/or mobility.

Capnographs typically serve to provide, three main outputs, which shed light on the patient's ventilation status:

a) End tidal CO₂ concentration (EtCO₂)—the highest concentration of CO₂ at the end of the exhalation stage, typically the highest CO₂ concentration measured during the last 30 seconds of monitoring;
b) A CO₂ concentration wave pattern as a function of time, indicative of the breath cycles (CO₂ waveform);

### c) Respiration Rate (RR).

The dual sensor breath monitoring devices, disclosed herein, include at least a flow sensor and a CO₂ sensor, the CO₂ sensor is configured to operate or inherently operates at low power. The flow sensor is configured to monitor “fast changing” parameters relating to the patient's respiratory status, including respiration rate and identification of apnea events, as well as to define initiation and ending of inhalation and exhalation periods. The CO₂ sensor, on the other hand, is configured to measure the “slower changing” CO₂ concentration in the patient's breath. The main use of the CO₂ concentration measurements is to confirm the ventilation efficiency of the patient and the patient's ability to maintain CO₂ concentrations that ensure a correct body pH, and these are not fast changing processes.

The breath monitoring device further includes a processor configured to integrate the measurements obtained from the flow sensor and the CO₂ sensor and to determine the respiratory status of the patient based on the integration. Measurements obtained from the flow sensor may be used as input for operation of the CO₂ sensor or calculation of the CO₂ concentration in the patient's breath. For example, during intermittent CO₂ sampling the OFF time of the CO₂ sensor may be determined based on the respiration rate (RR) measured using the flow sensor. In certain embodiment, the processor may use information from the flow sensor (e.g., information associated with the beginning and end of the patient's breath cycle) to generate representative CO₂ concentration waveforms having a suitable resolution for determining a concentration of CO₂ in the patient's breath with accuracy compared to CO₂ concentration waveforms generated using only information obtained from a CO₂ sensor operating at low power, which have not have a suitable resolution. Accordingly, the output of both the flow sensor and the CO₂ sensor may be integrated to optimize the information obtained from each breath in the patient's breath cycle and provide a better understanding of the patient's respiratory status using information obtained from sensors operating at low power. The breath monitoring solutions, provided herein, thus do not require the use of fast response CO₂ monitoring technologies and measurements, like those used in existing capnography, since the fast responses required for detecting events like apnea are achieved by the use of the flow sensor.

According to some embodiments, as used herein, the term “response time” of capnographs may refer to the delay caused by transit time, i.e. the time taken for a breath sample to travel (“transit”) from the sampling inlet to the sampling cell and/or the time required for an analyzer to “reset”, i.e. to prepare for a subsequent measurement.

According to some embodiments, as used herein, the term “rise time” may refer to the time required for the analyzer (capnograph) to respond once CO₂ has entered the sampling cell.

According to some embodiments, the term response time may include the term rise time and may thus refer to the overall time of measurement.

According to some embodiments, the processor may be configured to provide a single integrated value indicative of the patient's ventilatory status based on the measurements obtained from the flow sensor and the CO₂ sensor. According to some embodiments, the processor may be configured to provide an estimated CO₂ waveform based on the measurements obtained from the flow sensor and the CO₂ sensor. According to some embodiments, the processor may be configured to determine the clinical condition of the patient's lungs from a mechanical viewpoint, based on the measurements obtained from the flow sensor and the CO₂ sensor. Additionally or alternatively, the processor may be configured to provide clinical data relating to the patient's respiratory condition based on one or more parameters extracted from the flow waveform. According to some embodiments, the one or more parameters extracted from the flow waveform may include slope, area under curve, frequency, inhalation to exhalation ratio (I:E), repeatability, or any other suitable parameter or combination of parameters. Each possibility is a separate embodiment. Generally, a CO₂ concentration waveform obtained using data collected with a fast response time CO₂ sensor may provide information regarding changes in CO₂ concentration as a function of time during the breath cycle. However, when using the slow response time CO₂ sensor disclosed herein, changes in the CO₂ concentration as a function of time may be difficult to determine. A flow waveform generated from data obtained from the flow sensor is representative of mechanical functioning of the patient's breath and ventilation. Therefore, the flow waveform may be used to determine the CO₂ concentration as a function of time during the patient's breath cycle when intermittent CO₂ measurements are obtained using a slow response time CO₂ sensor.

Additionally or alternatively, the processor may be configured to provide a medical recommendation based on the determined respiratory status of the patient. Additionally or alternatively, the processor may be configured to display the single integrative value, the estimated CO₂ waveform, the medical recommendation, and/or any other suitable value determined based on the integration of the measurements obtained from the flow sensor and the CO₂ sensor on a display.

According to some embodiments, the breath-monitoring device may further include at least one additional sensor, such as at least 1, 2, 3, 4 or more additional sensors. Each possibility is a separate embodiment. According to some embodiments, the at least one additional sensor may include a photoplethysmograph (PPG) sensor, ECG sensor, temperature sensor, EOG sensor, pupil diameter monitor, EEG sensor, FEMG sensor, EMG sensor, EGG sensor, or any other suitable sensor. Each possibility is a separate embodiment.

According to some embodiments, the dual sensor breath monitoring devices disclosed herein may be suitable for use in neonatal, infants, children and adults. Each possibility is a separate embodiment.

According to some embodiments, the dual sensor breath monitoring devices disclosed herein may obviate the need for a pneumatic system for sampling the patient's breath and may be used without a pneumatic system for sampling the patient's breath.

According to some embodiments, the dual sensor breath monitoring devices disclosed herein may be suitable for use in out-of-hospital settings, such as in ambulances, field hospitals, and the like.

According to some embodiments, the dual sensor breath monitoring devices may enable at least 50, 60, 70, 80 percent or more power save as compared to “fast response” (25-100 millisecond) CO₂ sensors, and hence reduced cost. Each possibility is separate embodiment.

According to some embodiments, the flow sensor may be a pressure type transducer, preferably a differential pressure transducer measuring the pressure drop or differential pressure across a resistance to flow. A non-limiting example of pressure type transducers includes fiber-optic extrinsic Fabry-Perot interferometers (EFPI), such as diaphragm-based EFPI pressure sensors and white light based EFPI-sensors. Another non-limiting example of pressure type transducers includes piezo-resistors arranged in a Wheatstone bridge configuration.

According to some embodiments, the flow sensor may be a thermal type transducer, such as, but not limited to, hot wire anemometers measuring the cooling of a heated wire due to airflow passing around the wire, thermopiles (e.g. thermal mass flow sensors measuring temperature changes in the voltages of thermopiles caused by flow) or other suitable thermal type transducers. Each possibility is a separate embodiment.

According to some embodiments, the flow sensor may be a displacement based flow sensor, such as, but not limited to, ultrasonic dopplers measuring the frequency shift of an ultrasonic beam as it passes through gas flow, vortex shedding sensors counting the number of vortices that are shed as gas flows past a strut placed in the flow stream, Time of Flight meters, such as ultrasonic flow meters measuring the time difference between an ultrasonic pulse sent in the flow direction and an ultrasound pulse sent opposite the flow direction, or any other suitable displacement based flow sensor. Each possibility is a separate embodiment.

According to some embodiments, the CO₂ sensor may include a blackbody, a molecular correlation spectroscopy (MCS) source (also referred to herein as an IR lamp), an IR LED, an acoustic sensor, colorimetric sensor, transcutaneous CO₂ sensor, a PPG sensor, or any combination thereof. Each possibility is a separate embodiment.

According to some embodiments, the CO₂ sensor may include (or be) a CO₂ sensor having fast response capabilities, such as MCS sensors and certain blackbody-based technologies, operating in a power save mode.

According to some embodiments, a CO₂ sensor includes (or is) an infrared (IR) lamp. The lamp generates, utilizing an MCS source that operates at room temperature, CO₂ specific radiation only enabling an inherent fast response time: this fast response time is limited mainly by the time required for excited molecules in the radiating source to lose their energy as photons, a process taking a millisecond or two and is sufficiently fast in order to provide fast and accurate capnography. The main drawback of the IR specific lamp relates to energy waste. However, if the IR lamp is coupled with a flow sensor, the mode of operation may be altered so as to reduce energy consumption.

According to some embodiments, reduced power consumption may be achieved by activating the IR lamp intermittently. According to some embodiments, intermittent operation of the CO₂ sensor may include turning on the lamp for a first period of time and subsequently turning the lamp off for a second period of time, wherein the second period of time is longer than the first period of time. According to some embodiments, the duration of activation may be predetermined. According to some embodiments, the duration of activation may be determined based on the respiration rate measured by the flow sensor, e.g. sufficient to capture two or three entire breath cycles as determined by the flow meter. For example, if a 15 breaths-per-minute (bpm) respiration rate is determined, the lamp may be turned on for a first predetermined period of time, e.g. about 5 seconds, about 7 seconds, about 10 seconds, about 12 seconds, about 15 seconds, or any other suitable amount of time within a range of about 5-20 seconds. Each possibility is a separate embodiment. Subsequently, the lamp may be turned off for a second period of time (e.g. about 25 seconds, about 30 seconds, about 40 seconds, about 50 seconds, about 60 seconds, about 120 seconds or any other suitable amount of time within the range of about 25-120 seconds). The first and second periods of times may be updated based on the condition of the patient and/or one or more breath related parameters (e.g., respiration rate, CO₂ concentration, etc.) is at a desired level. For example, if the patient is having apnea or asthma symptoms, the first time period may be increased such that the CO₂ sensor is ON and more measurements are obtained. As the patient's condition improves, the first time period may be decreased. Similarly, if the patient achieves a desired respiration rate, the second time period may be increased. Each possibility is a separate embodiment. According to some embodiments, such intermittent operation may enable at least 50, 60, 70, or 80 percent power save, and hence reduced cost associated with operating the breath monitoring device. Each possibility is separate embodiment.

According to some embodiments, as used herein, the term “about” may refer to ±10, ±5, ±2, or ±1 percent. Each possibility is a separate embodiment.

According to some embodiments, reduced power consumption may be achieved by activating the lamp continuously, but with a lower than usual modulation frequency and duty cycle. According to some embodiments, a reduced modulation frequency may refer to a modulation frequency below 20 Hz, below 15 Hz, below 10 Hz, or any other modulation frequency within the range of 5-20. Each possibility is a separate embodiment. According to some embodiments, a reduced duty cycle may refer to a duty cycle below 50, below 40, below 30, below 20, or below 15 percent, or any other duty cycle within the range of 5-40 percent. Each possibility is a separate embodiment. As a non-limiting example, the lamp may be activated at a 4 Hz to 8 Hz modulation frequency with a 10 percent duty cycle. According to some embodiments, the lower than usual modulation frequency and duty cycle may enable at least 50, 60, 70, or 80 percent power save, and hence reduced cost. Each possibility is a separate embodiment.

According to some embodiments, the IR lamp may also be operated in mixed operation mode. That is, in the mixed operation mode, the IR lamp may operate at a first frequency and then switch to a second frequency that is different from (e.g., less than) the first frequency. For example, the lamp may operate at an optimum frequency (e.g. 20 to 40 Hz, 50 percent duty cycle) for a first period of time (e.g. 10 seconds), and at a lower frequency (e.g. 4 to 8 Hz, 10 percent duty cycle) for a subsequent second period of time (e.g. 50 seconds).

According to some embodiments, the modulation frequency may be determined based on the respiration rate detected by the flow sensor. For example, at higher respiration rates, modulation frequencies below 8-10 Hz may be too low. The respiration rate, as collected with the flow sensor, may thus be used to find (e.g., determine using the processor) the modulation frequency providing optimal power efficiency, while ensuring accurate data.

It is understood that the power save operation mode of the lamp provides fewer measurement points from which a CO₂ waveform may be built, and hence provides a poorer resolution compared to system operating in normal mode (e.g., not a power save mode). However, for normal adult breath rates, i.e. approximately 15 bpm, with exhalation period of 2 seconds duration, reconstruction of the waveform is still achievable. Moreover, the lamp, operating at low power, is sufficient for calculating EtCO₂ and even for providing usable information on the waveform shape during adult and pediatric monitoring. Moreover, low power consumption may enable breath monitoring in environments in which often no breath monitoring is performed, such as during transport, especially in the case of neonates, which are the main type of patients with high respiratory rates.

According to some embodiments, a CO₂ sensor includes (or is) a black body IR source. Blackbody IR sources use power to raise the temperature of the blackbody so as to obtain sufficient emission of radiation. The main electrical disadvantages of thermal emitters are their slow speed (compared to diode sources) and high drive power requirements. The time needed to raise the temperature of the blackbody depends on its size, where a small mass will have less inertia, but on the other hand, a reduced mass emits less light energy for any given temperature increase. Hence, when using a blackbody as an IR source, there is a tradeoff between power (and hence sensitivity) and response time. In order to improve the response time, the blackbody elements are being developed having a decreased size (MEMS), but resultantly, their emission is reduced. This dictates either an increased power input to ensure the quality of the measurements or a reduced sensitivity in favor of fast response time.

The main optical disadvantage of blackbodies is their wide, continuous spectral range, from which only a small part is useful for CO₂ detection. This directly reduces detection sensitivity, dictating a much longer optical path-length and hence increased response time.

However, when combining the blackbody with the flow sensor, as disclosed herein, the fast response time is not a prerequisite, and increased sensitivity (due to larger delta temperatures of the heated, emitting element, and/or due to using larger sized elements), may be achieved. According to some embodiments, the delta temperatures of the heated, emitting element may be in the range of 100-500, 150-500, 150-350, 175-500, 175-350 degrees Celsius, or any other suitable range.

In addition, overall power consumption may be reduced by using short ON time periods, i.e. low duty cycles or lower frequencies with low duty cycles, as essentially described above with regards to the IR lamp.

According to some embodiments, a CO₂ sensor includes (or is) an infrared light emitting diode (IR LED).

The power efficiency and response time of LEDs are highly conducive and appropriate for capnography, though they still suffer from optical power output sensitivity and central wavelength movement in response to temperature changes. This deficiency is typically solved by coupling the LEDs with a Peltier element or other temperature stabilizer, resulting in increased power consumption. Moreover, as in the case of blackbodies, the broad range of wavelengths emitted dictate a longer optical path, and consequently results in slower response times, insufficiently for high-resolution capnography. Though, this limitation may be resolved if the IR LED is partnered with a flow sensor, as disclosed herein, thereby obviating the need for fast response times.

According to some embodiments, the CO₂ sensor may be or include a slow response sensor, such as, but not limited to, an acoustic sensor, a calorimetric sensor, a transcutaneous sensor, or any combination thereof. Each possibility is a separate embodiment.

According to some embodiments, the CO₂ sensor includes (or is) an acoustic sensor configured to measure changes in the speed of sound as a function of the gas components in the surrounding media. The technology is based upon generating an oscillating motion of gas in a (optionally closed) cavity positioned between a transmitter and receiver, which requires little power for operation. The resonance and its frequency measured are related to the actual gasses in the cavity and may thus be used to identify the gas concentrations.

The technology requires a cavity of recognizable volume in order to measure the gas mixture resonance frequency as well as time to scan the frequencies in order to find the resonance, both limiting the response time of the measurements.

However, the dual sensor breath monitoring device disclosed herein, in which the CO₂ sensor, here the acoustic sensor, is partnered with a flow sensor, diminishes the disadvantage of utilizing such slow response sensors in breath monitoring devices.

According to some embodiments, the CO₂ sensor includes (or is) a colorimetric sensor.

Colorimetric based CO₂ measurements are based upon the fact that when CO₂ comes in contact with an aqueous solution, the hydrogen ion concentration increases, thus changing the pH of solutions. In effect, membranes and dyes may be used in order to induce color changes correlative to changes in CO₂ concentration. According to some embodiments, a light source and appropriate detectors with color signal processing etc. may be added to the sensor, thereby obtaining a basic colorimetric CO₂ sensor, which requires very little power.

The major drawback of calorimetric CO₂ monitoring is its relatively long response time, usually in the order of seconds. Though progress has been made in developing membranes with dyes and processing, which provide faster response times, the response time is typically still considered too slow (e.g., approximately 500 msec), and improvements in the response time are typically achieved at the expense of accuracy and repeatability.

However, partnering the colorimetric technology with a flow sensor, as disclosed herein, relieves the constraints of using faster response dyes and membranes, and the colorimetric membrane can be used for just providing periodic CO₂ concentration values.

According to some embodiments, optical fibers may be used to transmit signals between optical sources like LED's and the colorimetric sensor. According to some embodiments, the colorimetric sensor can be positioned in the patient airway path of intubated as well as non-intubated patients. According to some embodiments, the reflected light, may be transmitted via the optical fibers to an appropriate color detector, such as, but not limited to, an RGB detector. According to some embodiments, the flow sensor utilized may be a fiber optic sensor (e.g. a fiber-optic extrinsic Fabry-Perot interferometers (UPI)), such that both the colorimetric CO₂ sensor and the Fabry-Perot flow sensor, use optical fibers with minor power consumption with accompanying small battery needs and size. According to some embodiments, the entire sensor may be positioned at a convenient location on the patient, such as, but not limited to, on/in/behind the patient's ear, with fiber optics directed to an appropriate position in proximity to the patient's airway for measuring both flow and CO₂ concentrations. According to some embodiments, this would enable utilizing a (single) light detector (e.g. RGB detector) for both the flow and the CO₂ concentration measurements and will provide a simple, portable, and power efficient ventilation monitor. According to some embodiments, using fiber optics for both the CO₂ concentration measurement and the flow measurement may obviate the need for a pneumatic system for sampling the patient's breath.

Additionally or alternatively other types of flow sensors may be utilized in conjunction with the calorimetric CO₂ sensor, through dictating separate types of means for transmitting the signals, i.e. gas tubes, or conducting wires.

According to some embodiments, the CO₂ sensor may be configured to measure arterial CO₂ concentrations.

According to some embodiments, the CO₂ sensor includes (or is) a transcutaneous CO₂ sensor. Transcutaneous sensors typically induce hyperperfusion of the capillaries by increasing the local temperature of the skin at the sensor site. The externally applied heat alters the solubility of CO₂ in the blood and increases the metabolic rate of the skin by approximately 4-5 percent for every degree Celsius, resulting in local production of CO₂. According to some embodiments, the transcutaneous sensor includes an electrode, usually a Severinghaus electrode, which enables determination of the CO₂ concentration electrochemically, usually by a change in pH of an electrolyte solution. It is widely acknowledged that continuous heating of the skin may lead to discomfort at the skin, particularly in patients with thin, sensitive, or damaged skin. Changing sites as often as every 2 hours is therefore often necessary/required, especially in small premature neonates, and lower than optimal temperatures are often applied, despite the fact that the sensitivity and accuracy of the measurements may be compromised.

According to some embodiments, coupling the transcutaneous CO₂ monitoring technology with a fast flow sensor, as disclosed herein, permits discontinuous operation of the transcutaneous sensor. The intermittent rather than continuous use of the sensor enables heating the skin to a preferred temperature range without causing discomfort at the skin thereby improving its sensitivity and accuracy.

According to some embodiments, the coupling of the flow sensor and the transcutaneous CO₂ sensor may provide indications regarding the lung and ventilation activity, which is essentially bypassed when using the transcutaneous CO₂ sensor alone, as the readings only represent the concentration of CO₂ as measured in the blood.

According to some embodiments, the CO₂ sensor includes a PPG sensor configured to determine the patient's pulse waveform and a processing unit, the processing unit configured to indirectly measure the patient's arterial CO₂ concentration (PaCO₂), based on a resistance to blood flow as determined from a shape of the pulse waveform obtained from the PPG sensor.

According to some embodiments, the coupling of the flow sensor and the PPG sensor (configured to measure arterial CO₂ concentrations) may provide indications regarding the lung and ventilation activity, which is essentially bypassed when using the PPG sensor alone, as the readings only represent the concentration of CO₂ as measured in the blood.

According to some embodiments, there is provided a method for monitoring a patient's respiratory status, the method including obtaining a respiration rate and/or indications of an apnea event from a flow sensor; obtaining a CO₂ concentration in the patient's breath from a CO₂ sensor, the CO₂ sensor configured to operate at low power; integrating, using a processor, the measurements obtained from the flow sensor and the CO₂ sensor; and determining the respiratory status of the patient based on the integration. According to some embodiments, the method may be computer implemented.

According to some embodiments, the flow sensor may be any flow sensor configured to measure fast changing parameters in the patient's breath, as essentially described herein.

According to some embodiments, the CO₂ sensor may be any CO₂ sensor configured to measure a concentration of CO₂ in the patient's breath, and which is configured to work at low power or inherently requires low power, as essentially described herein.

According to some embodiments, determining the respiratory status of the patient may include providing a single integrative value indicative of the patient's respiratory status. According to some embodiments, determining the respiratory status of the patient may include providing an estimated CO₂ waveform.

According to some embodiments, the method may further include providing a medical recommendation based on the integrative measurements.

According to some embodiments, the method may further include displaying the determined respiratory status, the single inactive value indicative thereof, the estimated CO₂ waveform, the medical recommendation and/or any other suitable parameter/value on a display. Each possibility is a separate embodiment.

According to some embodiments, there is provided a processing unit configured to determine a patient's respiratory status, the processing unit configured to obtain a respiration rate and/or indications of an apnea event from a flow sensor; obtain a CO₂ concentration from a CO₂ sensor, wherein the CO₂ sensor is configured to operate at low power; integrate the obtained measurements and determine the respiratory status of the patient based on the integration.

According to some embodiments, the flow sensor may be any flow sensor configured to measure fast changing parameters in the patient's breath, as essentially described herein.

According to some embodiments, the CO₂ sensor may be any CO₂ sensor configured to measure a concentration of CO₂ in the patient's breath, and which is configured to work at low power or inherently requires low power, as essentially described herein.

According to some embodiments, the processing unit may be configured to provide a single integrative value indicative of the patient's respiratory status. According to some embodiments, the processing unit may be configured to provide an estimated CO₂ waveform.

According to some embodiments, the processing unit may be configured to provide a medical recommendation based on the integrative measurements.

According to some embodiments, the processing unit may be configured to display the determined respiratory status, the single inactive value indicative thereof, the estimated CO₂ waveform, the medical recommendation and/or any other suitable parameter/value on a display. Each possibility is a separate embodiment.

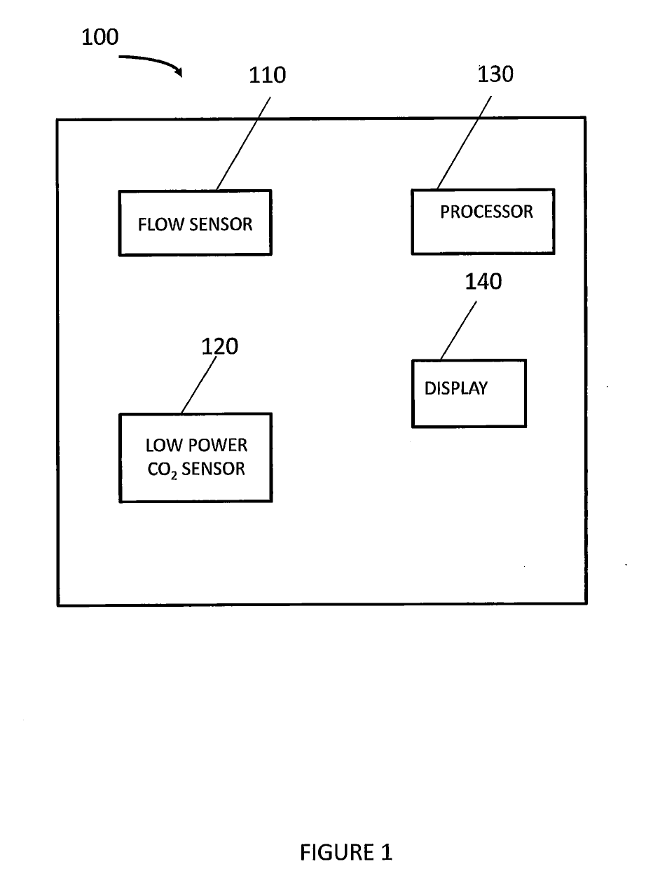

Reference is now made to FIG. 1, which illustrates a dual sensor breath monitoring device 100 including a flow sensor 110 and a low power CO₂ sensor 120. Flow sensor 110 is configured to monitor “fast changing” parameters relating to the patient's respiratory status including respiration rate and identification of apnea events as well as to define initiation and ending of inhalation and exhalation periods. Flow sensor 110 may be any type of sensor capable of monitoring a patient's breath flow, as essentially described herein. CO₂ sensor 120 is a CO₂ sensor, which is either configured to operate in a power saving mode, at the expense of response time or a CO₂ sensor having an inherent slower response time, which are not typically considered suitable for capnography. For example, the CO₂ sensor 120 may be a calorimetric, an acoustic, a transcutaneous, or any other suitable type of power saving CO₂ sensor. The CO₂ sensor 120 is thus configured to provide periodic/intermittent evaluations of the “slower changing” CO₂ concentration in the patient's breath. The main use of the CO₂ concentration measurements is to confirm the ventilation efficiency of the patient and the patient's ability to maintain CO₂ concentrations that ensure a correct body pH, and these are not fast changing processes and may be any of the CO₂ sensors disclosed herein. Breath monitoring device 100 further includes a processor 130 configured to integrate the measurements obtained from flow sensor 110 and CO₂ sensor 120 and to determine the respiratory status of the patient based on the integration. Processor 130 may be configured to provide a single integrative value indicative of the patient's respiratory status and/or indicative of the condition of the patient's lungs from a mechanical viewpoint, based on the integration of the measurements obtained from flow sensor 110 and the CO₂ sensor 120. Additionally or alternatively, processor 130 may be configured to construct an estimated CO₂ waveform based on the measurements obtained from the flow sensor 110 and the CO₂ sensor 120. Processor 130 may be configured to display the single integrative value and/or the estimated CO₂ waveform on a display 140.

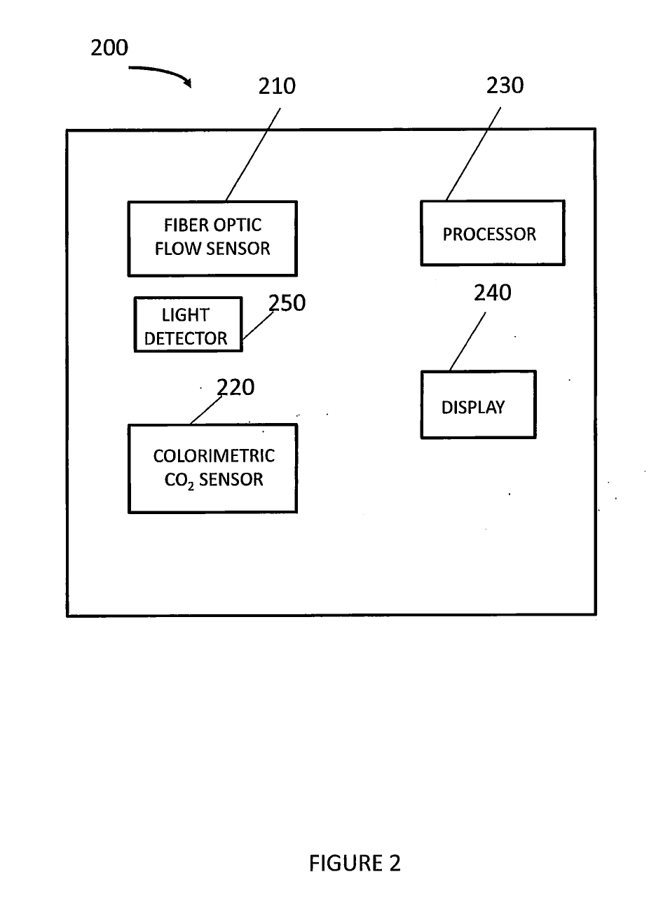

Reference is now made to FIG. 2, which illustrates a non-limiting example of an all optic, dual sensor, breath monitoring device 200 including a fiber optic flow sensor 210 and a calorimetric CO₂ sensor 220. Fiber optic flow sensor 210 is configured to monitor “fast changing” parameters relating to the patient's respiratory status including respiration rate and identification of apnea events as well as to define initiation and ending of inhalation and exhalation periods. Calorimetric CO₂ sensor 220 inherently has slower response times, but enables to confirm the ventilation efficiency of the patient and the patient's ability to maintain CO₂ concentrations that ensure a correct body pH, as these are not fast changing processes. Advantageously, a (single) light detector 250 of breath monitoring device 200 may both enable monitoring changes in CO₂ concentrations based on color changes obtained by the calorimetric CO₂ sensor 220 as well as changes in flow monitored by the flow sensor 210, thereby providing a simple, portable, power efficient and low cost ventilation monitor. Breath monitoring device 200 further includes a processor 230 configured to integrate the measurements obtained from the fiber optic flow sensor 210 and the calorimetric CO₂ sensor 220 and to determine the respiratory status of the patient based on the integration. Processor 230 may be further configured to provide a single integrative value indicative of the patient's respiratory status and/or indicative of the condition of the patient's lungs from a mechanical viewpoint, based on the integration of the measurements obtained from the fiber optic flow sensor 210 and the calorimetric CO₂ sensor 220. Additionally or alternatively, the processor 230 may be configured to construct an estimated CO₂ waveform based on the measurements obtained from fiber optic flow sensor 210 and calorimetric CO₂ sensor 220. The processor 230 may be configured to display the single integrative value and/or the estimated CO₂ waveform on a display 240.

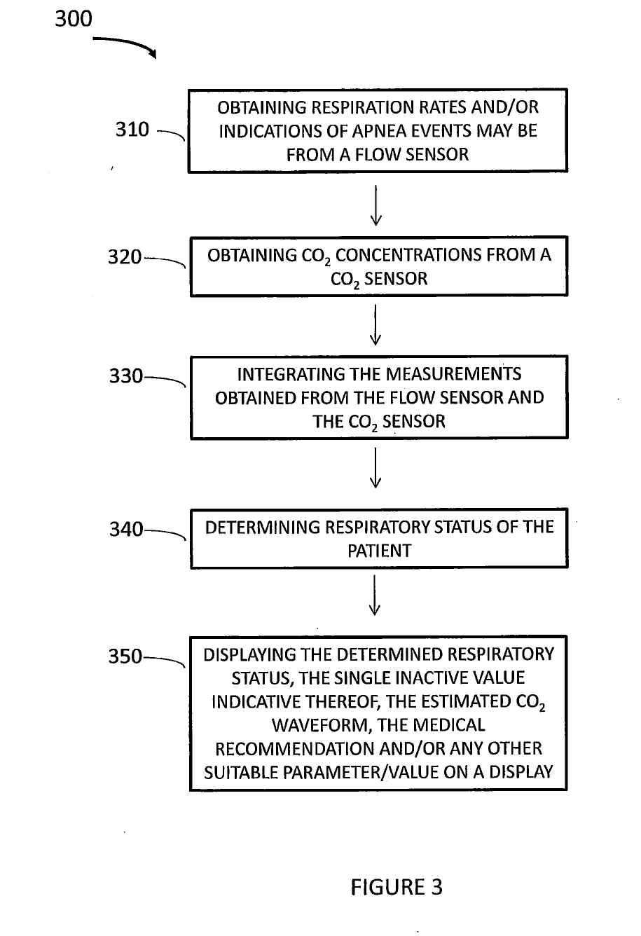

Reference is now made to FIG. 3, which an illustrative flowchart of a method 300 for determining a patient's ventilatory status using a dual sensor breath monitoring device (e.g., the device 100 of FIG. 1 or the device 200 of FIG. 2), according to some embodiments. In step 310 of the method, a respiration rate and/or indications of apnea events may be obtained from a flow sensor. The flow sensor may be any flow sensor configured to measure fast changing parameters in the patient's breath, as essentially described herein.

In step 320 of the method, a CO₂ concentration is obtained from a CO₂ sensor, the CO₂ sensor configured to operate at low power. The CO₂ sensor may be any CO₂ sensor configured to measure a concentration of CO₂ in the patient's breath, and which may work at low power or inherently requires low power, as essentially described herein.

In step 330 of the method, the measurements obtained from the flow sensor and the CO₂ sensor are integrated, and in step 340 the respiratory status of the patient is determined based on the integration. As described herein, determining the respiratory status of the patient may include providing a single integrative value indicative of the patient's respiratory status. Additionally or alternatively, determining the respiratory status of the patient may include providing an estimated CO₂ waveform.

According to some embodiments, the method may further include an additional step 340 of providing a medical recommendation based on the integrative measurements.

According to some embodiments, the method may further include an additional step 350 of displaying the determined respiratory status, the single inactive value indicative thereof, the estimated CO₂ waveform, the medical recommendation and/or any other suitable parameter/value on a display.

As used herein, the terms “patient” and “subject” may be interchangeably used and may refer to any subject undergoing breath monitoring.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

Unless specifically stated otherwise, as apparent from the following discussions, it is appreciated that, throughout the specification discussions, utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present disclosure may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present disclosure are not described with reference to any particular programming language. It will be appreciated that a variety of programming languages may be used to implement the teachings of the embodiments as described herein.

The embodiments may be described in the general context of computer-executable instructions, such as program modules, being executed by a computer. Generally, program modules include routines, programs, objects, components, data structures, and so forth, which perform particular tasks or implement particular abstract data types. The embodiments may also be practiced in distributed computing environments where tasks are performed by remote processing devices that are linked through a communications network. In a distributed computing environment, program modules may be located in both local and remote computer storage media including memory storage devices.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A breath monitoring device comprising
a flow sensor configured to measure a respiration rate of a patient and to identify apnea events in the patient's breathing;
a CO₂ sensor configured to operate at low power and to measure a CO₂ concentration in the patient's breath; and
a processor configured to integrate the measurements obtained from the flow sensor and the CO₂ sensor and to determine the respiratory status of the patient based on the integration.

2. The breath monitoring device of claim 1, wherein the CO₂ sensor comprises a blackbody, a molecular correlation spectroscopy (MCS) source, or an IR LED.

3. The breath monitoring device of claim 2, wherein the CO₂ sensor is configured to operate intermittently and/or to operate at a modulation frequency below 20 Hz and at a duty cycle below 50 percent.

4. The breath monitoring device of claim 3, wherein the CO₂ sensor operates intermittently and wherein the intermittent operation of the CO₂ sensor comprises turning the device and/or the CO₂ sensor ON for a first period of time and turning the device and/or CO₂ sensor OFF for a second period of time, wherein the second period of time is longer than the first period of time.

5. The breath monitoring device of claim 4, wherein the second period of time is at least twice the length of the first period of time.

6. The breath monitoring device of claim 4, wherein the first period of time is in the range of about 5-15 seconds and the second period of time is in the range of about 40-120 seconds.

7. The breath monitoring device of claim 4, wherein the processor is configured to change the first period of time, the second period of time, or both, based on a medical condition of the patient.

8. The breath monitoring device of claim 3, wherein the CO₂ sensor operates at a modulation frequency above 20 Hz and at a duty cycle above 40 percent during a first period of time and at a modulation frequency below 20 Hz and at a duty cycle below 40 percent during a second period of time, when in use, wherein the second period of time is longer than the first period of time.

9. The breath monitoring device of claim 2, wherein the CO₂ sensor operates at a modulation frequency below 20 Hz and at a duty cycle below 40 percent, when in use.

10. The breath monitoring device of claim 8, wherein the modulation frequency of the CO₂ sensor is determined by the respiration rate monitored by said flow sensor.

11. The breath monitoring device of claim 10, wherein the second period of time is at least twice the length of the first period of time.

12. The breath monitoring device of claim 10, wherein the first period of time is in the range of about 5-15 seconds and the second period of time in the range of about 40-120 seconds.

13. The breath monitoring device of claim 1, wherein the CO₂ sensor comprises an acoustic sensor configured to measure changes in the speed of sound in a breath sample obtained from said patient.

14. The breath monitoring device of claim 1, wherein the CO₂ sensor comprises a colorimetric sensor comprising a membrane and/or dye configured to change its color in response to a change in the pH of a breath sample obtained from the patient.

15. The breath monitoring device of claim 14, wherein the colorimetric sensor further comprises a detector configured to detect the color of the membrane and/or dye.

16. The breath monitoring device of claim 14, wherein the colorimetric sensor is configured to be positioned in the patient's airway.

17. The breath monitoring device of claim 1, wherein the CO₂ sensor comprises a transcutaneous CO₂ sensor.

18. The breath monitoring device of claim 1, wherein the transcutaneous CO₂ sensor is configured to operate periodically, thereby blocking heat irritation to the patient's skin.

19. The breath monitoring device of claim 1, wherein the CO₂ sensor comprises a PPG sensor configured to determine the patient's pulse waveform, wherein the processing unit is configured to indirectly measure the patient's arterial CO₂ concentration (PaCO₂), based on a resistance to blood flow as determined from a shape of the pulse waveform obtained from the PPG sensor.

20. The breath monitoring device of claim 1, wherein the flow sensor comprises a pressure type transducer, a thermal type transducer, or any combination thereof.

21. A method for monitoring a patient's respiratory status, the method comprising:
obtaining a respiration rate and/or indications of an apnea event from a flow sensor;
obtaining a CO₂ concentration in the patient's breath from a CO₂ sensor, the CO₂ sensor configured to operate at low power;
integrating, using a processor, the measurements obtained from the flow sensor and the CO₂ sensor; and
determining the respiratory status of the patient based on the integration.
